(19) United States
(12) Patent Application Publication
(10) Pub. No.: US 2018/0218781 A1
(43) Pub. Date: Aug. 2, 2018

(54) ELECTRONIC HEALTH RECORDS MANAGEMENT PLATFORM AND METHODS

(71) Applicant: OnService Solutions, LLC, Acton, MA (US)

(72) Inventor: Steven Pestka, Acton, MA (US)

(21) Appl. No.: 15/937,366

(22) Filed: Mar. 27, 2018

Related U.S. Application Data
(63) Continuation of application No. 14/210,074, filed on Mar. 13, 2014, now abandoned.
(60) Provisional application No. 61/784,285, filed on Mar. 14, 2013.

(51) Int. Cl. G16H 10/60

(52) U.S. Cl. CPC G16H 10/60; G06F 19/322

(57) ABSTRACT

Disclosed are systems and methods for electronic health record management and logging of hand-offs of patient care in a healthcare facility. The system may include a communication module for recording and time stamping a patient's clinical information; a hand-off logging module for recording information relating to the patient's hand-off from a first healthcare provider to a second healthcare provider, and for recording information relating to any subsequent hand-offs of the patient to other healthcare providers. The recorded hand-off information includes the names of the healthcare providers providing health services to the patient, and a time stamp associated with each hand-off, and the hand-off logging module further generates a hand-off log including the recorded hand-off information and the patient admission information. A hand-off threading module generates a hand-off thread that includes a respective clinical synopsis relating to the patient by each named healthcare provider associated with a recorded hand-off.

### RELATED APPLICATIONS

This application is a continuation of U.S. patent application Ser. No. 14/210,074, filed Mar. 13, 2014, and entitled “Electronic Health Records Management Platform and Methods”. U.S. patent application Ser. No. 14/210,074 claims priority to U.S. provisional application No. 61/784,285, filed Mar. 14, 2013, and entitled “Healthcare Workflow/EMR Management Platform”.

Each of the foregoing applications is incorporated herein by reference in its entirety.

### BACKGROUND

### Field of the Invention

The methods and systems disclosed herein generally relate to the management of healthcare patients and data relating to their treatment as they interact with a plurality of healthcare providers and/or healthcare locations, and the consolidation and presentation of such data for use by a plurality of healthcare providers and/or healthcare locations. The methods and systems relate more particularly to hand-offs of responsibility for patients among providers within a healthcare environment.

### Description of the Related Art

Electronic Health Records (EHRs) transpose traditionally paper records into digital format, such as provider orders, clinical documentation, pharmaceutical formularies, patient laboratory, and radiology results. There exist healthcare software applications that are specifically designed to streamline workflow for healthcare providers (physicians and non-physician providers) who care for patients in the inpatient setting. Such software focuses on the operations and workflow processes of inpatient providers. Fundamental functions of such applications may be actions such as logging patents by an inpatient provider (such as hospitalist, inpatient consultant, or designee) who require admission to the hospital or specialty consultation, distributing patients to inpatient providers, with incorporated logic and rules based on provider staffing, or transitioning a patient from the care of one inpatient provider to another inpatient provider, with an incorporated template and logic to ensure appropriate transfer of important clinical elements.

Inpatient providers typically document progress notes daily on every hospitalized patient. These progress notes contain information related to, for example, the patient's current symptoms, exam, laboratory and radiologic data. The progress notes also typically include sections titled “Impression” and “Plan.” The impression section reviews the provider's overall impression of the patient's clinical status and progress, and the plan reviews each medical problem and the corresponding plan. However, when an inpatient provider is ready to go off-service, they are required to provide an off-service hand-off to the next provider. The provider going off-service typically enters their current impression of the patient's status, and provides a list of relevant medical problems, but this information is typically fragmentary and lacks a summary of the patient's overall care to date, current status and proposed care plan. Therefore there is a need for a software tool that may provide a more effective, concise summary of providers' impressions and statements, and the problem-based plan that has evolved for a patient, to enable the healthcare provider who is coming onto service to quickly gain an understanding of the patient's issues so that they can provide uninterrupted care to the patient.

### SUMMARY

Provided herein are methods and systems for a healthcare workflow/EHR management platform that assist in the patient care hand-off between professionals at transition points in care, such as during and following a hospitalization. The platform may include a hand-off logging module, a hand-off survey module, a hand-off threading module, a communication and logging module, a readmission notification and survey module, a progress note creation tool and/or a hand-off auto-import module.

In embodiments, the hand-off logging module may include functionality that enables date and time stamping of hand-offs within an EHR system. In embodiments, when a hand-off occurs, and by whom, may be logged in the hand-off log. In embodiments, the hand-off survey module may include functionality providing a front-end administrative interface where hand-off surveys are created and where questions are formed. The survey displayed may include clinical information received at the time of hand-off. In embodiments, the survey module may include a survey database and may further include functionality allowing an administrative user to customize surveys. In embodiments, the hand-off thread module may include functionality allowing for the creation of an information thread of hand-off content. In embodiments, the communication and logging module may include functionality allowing the entering of an admitting clinical synopsis for a patient when that patient is admitted to the healthcare providing facility. The synopsis may be sent to one or more outpatient providers so they may be informed that a patient is admitted and receive a brief overview of the reason for admission. In embodiments, the readmission notification and survey module may work in conjunction with the hand-off log module to clearly identify what hospitalists were involved in a patient's care for a prior hospital stay.

In embodiments, an electronic health records (EHR) management platform may include a communication module for recording and time stamping communications that occur during a patient's admission in a healthcare facility, and a hand-off logging module for recording information relating to the patient's hand-off from receiving health services in the healthcare facility from a first healthcare provider (such as a medical doctor) to a second healthcare provider (such as another medical doctor) and for recording information relating to any subsequent hand-offs of the patient to other healthcare providers. The recorded hand-off information may include the names of the healthcare providers providing health services to the patient and a time stamp associated with each hand-off. The hand-off logging module may generate a hand-off log including the recorded hand-off information and the patient clinical information, and optionally patient discharge information. The platform may also include a hand-off threading module for generating a hand-off thread that includes a respective clinical synopsis relating to the patient by each named healthcare provider associated with a recorded hand-off, and a user interface that enables a user to view the patient's hand-off log and/or the patient's hand-off thread and/or a communication log generated by the communication module.

In embodiments, the communication module may be enabled to send communications regarding the patient's hand-off log and/or the patient's hand-off thread to an external healthcare provider, an external electronic health records system, and/or a quality monitoring module.

In embodiments, the EHR management platform may also include a progress note creation tool for recording a progress note of the patient by a named healthcare provider, and a hand-off thread export tool for exporting the recorded progress note of the patient to be incorporated in the hand-off thread.

In embodiments, the EHR management platform may include a survey module for generating surveys regarding the respective quality of each hand-off, wherein each survey is directed to a specific hand-off and the survey is communicated to the healthcare provider on the receiving end of the specific hand-off, and the survey module records and analyzes survey results.

In embodiments, the EHR management platform may include a survey module for generating a survey regarding readmission of a patient, wherein the readmission survey is directed to the discharging healthcare provider.

In embodiments, the healthcare facility may be a hospital, a skilled nursing facility, a rehabilitation facility, a long-term acute care facility, a post-acute care facility, or the like.

In embodiments, a method includes recording patient clinical information associated with a patient's admission to a healthcare facility, wherein the patient clinical information includes a time stamp (date and time) of the patient's admission; recording information relating to each hand-off of care of the patient in the healthcare facility from one healthcare provider to another healthcare provider, wherein the recorded hand-off information includes the names of the healthcare providers providing health services to the patient, and a time stamp associated with each hand-off; generating a hand-off log including the recorded hand-off information and the patient clinical information; generating a hand-off thread that includes a respective clinical synopsis relating to the patient by each named healthcare provider associated with each recorded hand-off; and communicating at least one of the hand-off thread and the hand-off log to a user, or communicating the patient clinical information to other providers, wherein all communications are entered in a communication log which includes the names of the sending provider and recipient provider, a time stamp associated with each communication, method of communication, and the patient clinical information.

In embodiments, the user includes at least one of an external healthcare provider, an external electronic health records system, and a quality-monitoring module.

In embodiments, the method further includes recording patient discharge information associated with the patient's discharge from the healthcare facility, wherein the patient discharge information includes a time stamp of the patient's discharge, and adding the patient discharge information to the hand-off log.

In embodiments, the method further includes generating a survey regarding the quality of a specific hand-off and communicating the survey to the healthcare provider on the receiving end of the specific hand-off.

In embodiments, the method also includes recording and analyzing survey results.

In embodiments, the method also includes generating a survey regarding readmission of the patient when the patient is readmitted to the healthcare facility and communicating the survey to the discharging healthcare provider.

These and other systems, methods, objects, features, and advantages of the present invention will be apparent to those skilled in the art from the following detailed description of the preferred embodiment and the drawings. All documents mentioned herein are hereby incorporated in their entirety by reference.

### BRIEF DESCRIPTION OF THE DRAWINGS

The systems and methods described herein may be understood by reference to the following figures:

FIG. 1A is a simplified diagram of an exemplary embodiment of an EHR Management Platform;

FIG. 1B is an example workflow using the embodiment of the EHR Management Platform depicted in FIG. 1A;

FIG. 1C depicts an embodiment of a hand-off logging module;

FIG. 2A depicts an embodiment of a hand-off survey module implemented in conjunction with a hand-off log;

FIG. 2B depicts an embodiment of a hands-off survey module front-end administrative interface;

FIG. 2C depicts an embodiment of a notification generated to remind a user to complete a survey and an embodiment of a hands-off survey module front-end administrative interface;

FIG. 3A depicts an embodiment of a hand-off threading module;

FIG. 3B depicts an embodiment of a hand-off form;

FIG. 4 depicts an embodiment a communication and logging module and an embodiment of a communication log;

FIG. 5A depicts an embodiment of a progress note creation tool;

FIG. 5B depicts an embodiment of a progress note and an embodiment of the hand-off auto-import module; and

FIG. 5C depicts an embodiment of a hand-off auto-import module.

### DETAILED DESCRIPTION

The present invention will now be described in detail by describing various illustrative, non-limiting embodiments thereof with reference to the accompanying drawings and exhibit. The invention may, however, be embodied in many different forms and should not be construed as being limited to the illustrative embodiments set forth herein. Rather, the embodiments are provided so that this disclosure will be thorough and will fully convey the concept of the invention to those skilled in the art. The claims should be consulted to ascertain the true scope of the invention.

Provided herein are systems and methods that streamline workflow for healthcare “providers” (both physicians and non-physician providers) who care for “inpatients,” patients cared for in a non-ambulatory healthcare facility. A non-ambulatory healthcare facility may refer to a hospital, skilled nursing facility, acute rehabilitation facility, rehabilitation facility, long-term acute care facility, or any post-acute care facility (the term “hospital” as used herein may represent any of these healthcare facilities except where context indicates otherwise). The systems and methods disclosed herein may improve current Electronic Health Records (EHRs) and their implementation. EHR, as used herein, includes electronic medical records, and other electronic data relating to a patient's health status, treatment, medications, course of care, treatment plan, and healthcare providers' notes and commentary relating to the same. The systems and methods disclosed herein may improve operational efficiency and workflow processes of inpatient providers by providing improvements to existing healthcare management software applications. The systems and methods disclosed herein may provide new metrics for provider groups or healthcare organizations to track provider, group or organizational performance.

Embodiments of the systems and methods disclosed herein may comprise a healthcare workflow/EHR management platform (the “platform”). In embodiments, the healthcare workflow management platform may comprise a hand-off logging module. A hand-off typically occurs when the first healthcare provider goes “off-service,” or ends a specified period of time caring for inpatients, and a second healthcare provider comes “on-service,” or begins a period of time caring for inpatients. Another important type of hand-off occurs when an on-service provider hands-off for a time-limited period to a “covering provider.” This type of hand-off to a covering provider may occur, for example, during overnight, weekend, or holiday periods. Such software further includes backend logic that assigns a provider who is receiving a hand-off as the “attending physician” or “responsible provider.” Other functions of existing EHRs may be electronic communication of triage, hand-off, and discharge data from an inpatient provider to a patient's primary care doctor or other provider who assumes responsibility for a patient's medical issues upon discharge from the hospital, enabling inpatient providers to electronically document a patient's progress and the clinical plan of care, displaying patient admissions from a specified timeframe, or displaying patients that are assigned to a specified user.

A patient hand-off may occur when one provider hands-off a patient to another provider. If a patient remains in the hospital or other healthcare facility for a long period of time, sometimes several hand-offs occur. Current hospital EHRs typically track information regarding other involved providers. This information is often disconnected, and is not logged in a cohesive or easily retrievable format. Further, information in current hospital EHRs does not typically include the actual date and time that the hand-off information was entered, but instead includes information based on an electronic order. Such an order is instead a transfer of care acknowledgement, which sometimes occurs several hours following the hand-off entry. Inpatient consultants typically have no manner of logging when one consultant goes off service, and when another consultant comes on service. Lastly, information about transfers of care among inpatient consultants is not currently logged at all in EHR systems.

Hand-offs from one provider to another provider are critically important transitions of care. Communicating a patient's active medical problems, pertinent medical history, incidental findings, and issues that require further workup or close monitoring are among the key elements of a good transition of care. Due to the inherent nature of a hand-off, where one provider goes off service and a different provider assumes care of a patient, transitions of care represent a vulnerable point in time when errors may occur in the management of a patient. The Joint Commission as well as various medical societies have recognized the importance of high quality hand-offs. In the prior art, transitions of care are either not standardized at all, or are standardized through a written or electronic template. There is, however, no way to measure the quality of hand-offs in an automated fashion. Embodiments of the systems and methods disclosed herein may allow for providers who receive hand-offs from another provider to rate the quality of every hand-off through an automated survey. Automated hand-off surveys provide frequent feedback on the quality of a provider's hand-offs. Since feedback based on these surveys is likely to have a positive effect on the quality of hand-offs, the systems and methods disclosed herein have the capacity to improve patient outcomes.

Provider-to-provider hand-offs typically occur outside the scope of an EHR. In cases where a hand-off occurs within an existing EHR, the hand-off is typically limited to a single, large text field or similar type of field. Such a field is overwritten the next time a hand-off occurs for a patient, and pre-existing hand-off content is not easily viewable by end-users. Frequently, this information is not retrievable at all, even from the back-end, by EHR technical users.

When patients are admitted to the hospital, the doctors that typically care for the patient in the outpatient setting need to remain updated. This is because hospitalists most frequently care for patients during the inpatient admission, as opposed to outpatient providers (outpatient providers are typically the primary care physician, nursing home/facility doctor, or specialists such as cardiologists).

In the conventional situation, a patient is “readmitted” to a hospital when they are hospitalized or discharged, and then require a second hospitalization within a specified period of time. This specified period of time can vary, but 30 days is frequently used to define a “readmission.” Readmissions have recently entered the healthcare spotlight. This is in part due to the fact that a readmission is often considered a measure of suboptimal quality of care. Further, Medicare is beginning to deny payment for particular types of readmissions. Hospitalists may not be aware that their patient was readmitted and do not typically have an opportunity to provide feedback on the reason for readmission. This opportunity for feedback may allow for insight and may help experts who are studying readmissions to better understand the reason for many readmissions, and to consider ways to impact the rate of readmissions. Current EHRs do not automatically alert providers who cared for a patient during a prior hospital stay that the patient was readmitted.

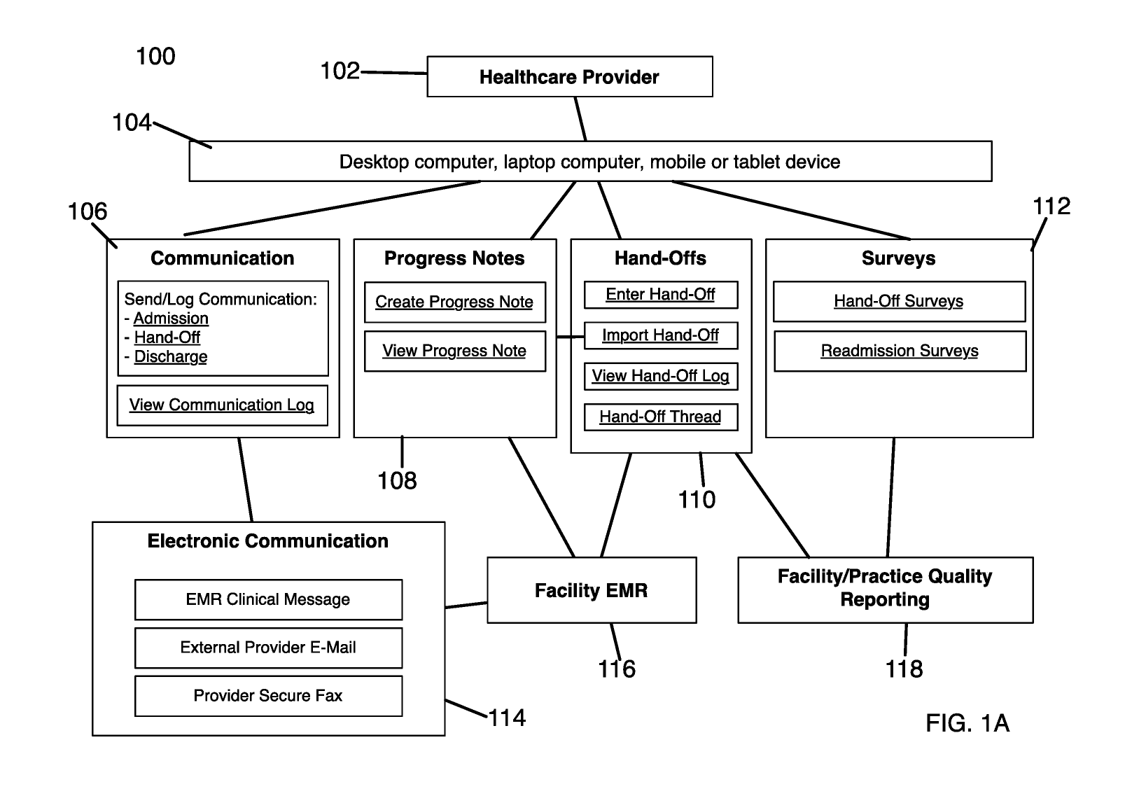

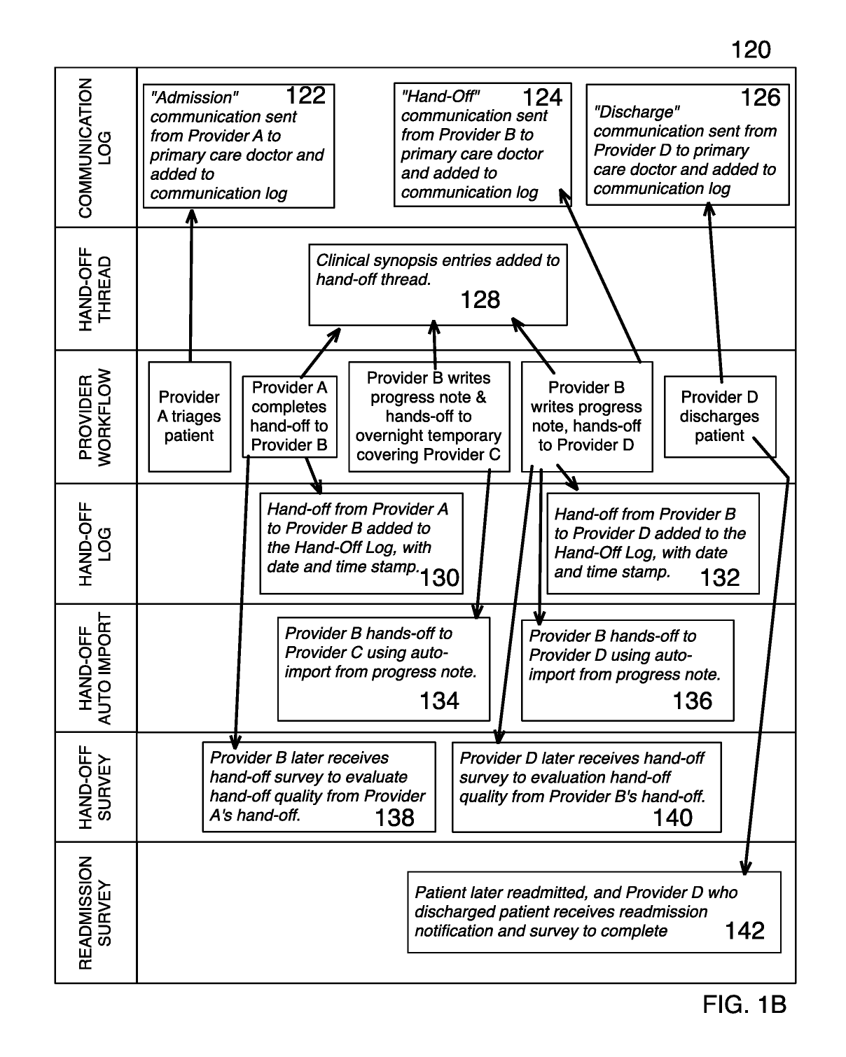

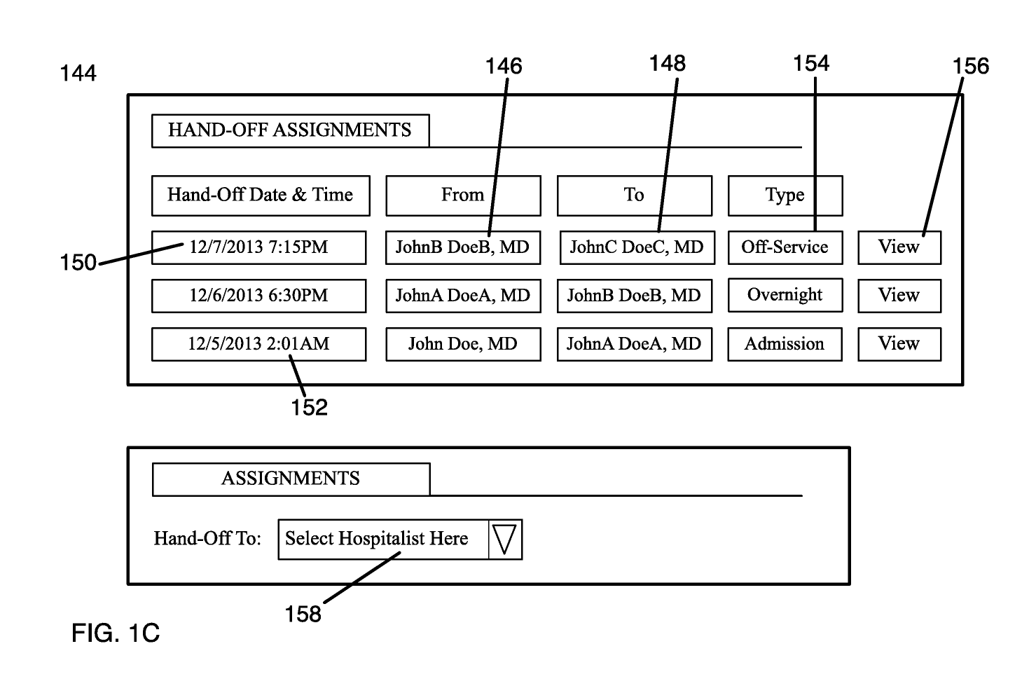

Referring to FIG. 1A, an overview of an exemplary embodiment of the healthcare workflow/EHR management platform 100 of the present disclosure is presented. A healthcare provider 102 may interact with the platform 100 through a device 104 to manage patient information. Device 104 may be a desktop computer, laptop computer, tablet, mobile device, or the like. Provider communications may be sent and logged in a communication module 106. Electronic notifications entered in the communication module 106 may be sent by an electronic communication module 114 such as via EHR clinical message (through an EHR interface), or may be sent to a provider by encrypted e-mail or secure fax 114. The communication module 106 may include immutable data, including information related to the sender, recipient, date/time, and content of the communication. The communication module 106 may also interface with a facility EHR system 116. Through a progress note creation tool 108, a provider caring for a patient in a healthcare facility may create a progress note and import information from the progress note automatically to a hand-off form of Hand-off module 110 in order to record a hand-off of a patient from one provider to another facility provider. When the hand-off form is completed, a hand-off occurs from one provider to another; this action may result in the addition of an entry to the hand-off logging module 110. Recorded information in the hand-off logging module includes the name of the sending provider, the receiving provider who is accepting the patient's care in the facility, and a time stamp (the date and time) of the hand-off. The hand-off information from one facility provider to another are also captured in a hand-off thread, an immutable thread of the clinical information from each hand-off along with the name of the entering provider and the date/time that the clinical information was entered. Progress notes may be uploaded to the facility EHR system 116; similarly, the clinical information contained in the hand-off thread and the data from the hand-off log may interface with the facility EHR system 116 (directly or via the communication module 106). Information from the hand-off thread and log may further supplement a healthcare facility's or provider practice's reporting, and as such may be used as a source for a quality data or provider performance reporting module 118.

A survey module 112 may be used to create hand-off surveys created at the time of a provider-to-provider hand-off, which allows the provider assuming the care of a patient in a facility to evaluate the quality of the hand-off information and clinical synopsis entered by the initial caring provider. Survey module 112 may also be used to create readmission surveys, such that when a patient is readmitted to the hospital, the provider who discharged the patient from the facility may be notified of the readmission and presented with a readmission survey, allowing them to enter the possible contributing causes of the readmission and other relevant information. An admitting provider in the facility at the time of the readmission may also receive a readmission survey.

FIG. 1B presents a simplified example workflow of providers within a facility interacting with the exemplary platform of FIG. 1A. First, when a provider initially triages a patient for admission to a facility, clinical information regarding the patient is logged at the time of triage via the communication module, and that information can be sent to one or more providers who care for the patient in the outpatient setting (providers who care for the patient when they are not in the facility) at a step 122. This communication is entered in a communication log as an “Admission” communication, and is date and time stamped at step 122.

Next, Provider A, caring for a patient in the facility, hands-off the care of the patient to Provider B. With this hand-off, an entry is added to the hand-off log at a step 130, and a clinical synopsis generated by Provider A is added to a hand-off thread at a step 128. This hand-off also triggers the creation of a Hand-Off Survey, which Provider B receives at a later time, at step 138.

Next, Provider B writes a progress note regarding the patient and hands off care of the patient to Provider C. In this example, Provider C is temporarily caring for the patient overnight. At a step 134, the provider uses the Hand-Off Auto Import function to automatically populate the required fields for the hand-off to Provider C from the progress note. This hand-off from Provider B to Provider C also generates an entry in the hand-off thread at a step similar to step 128.

Provider B writes a subsequent day's progress note, hands off care of the patient to Provider D. Again, Provider B uses the Hand-Off Auto Import function to automatically populate the required fields for the hand-off of the care of the patient to Provider D from the progress note at a step 136. Provider D subsequently assumes the care of the patient from Provider B. This hand-off from Provider B to Provider D also adds Provider B's clinical synopsis to the hand-off thread at a step similar to step 128. At a step 132, the Hand-Off information from Provider B to Provider D is recorded in the hand-off log, and a notification containing the clinical synopsis is generated and sent to the patient's primary care provider and added to the communication log at a step 124. This communication may be specified in the communication log as a “Hand-Off” communication. This hand-off also generates another Hand-Off survey, this time for Provider D to evaluate the quality of the hand-off from Provider B at a step 140.

Next, Provider D discharges the patient from the health care facility. At the time of discharge, Provider D notifies the primary care doctor that the patient has been discharged and includes the relevant clinical information in the communication; this notification is added to the communication log as a “Discharge” communication at a step 126. If the patient is later readmitted to the hospital, Provider D receives a notification that the patient was readmitted and a survey related to the readmission to be completed at a step 142.

In embodiments and referring to FIG. 1C, the hand-off logging module may comprise functionality enabling the entry of each hand-off and date and time stamping of all hand-offs within an EHR system that may occur over the course of treatment for a plurality of patients. In embodiments, contents of the hand-off log 144 may comprise entries in the log such as, but not limited to: 1. “Hand-off From” provider 146 (or sending provider), 2. “Hand-Off To” provider 148 (or receiving provider), 3. Date of hand-off 150, and 4. Time of hand-off 152. In embodiments, a new hand-off may be generated and logged when, after updating the clinical information for the hand-off, the sending provider selects the receiving provider in the hand-off form 158. When the form is saved, a hand-off occurs and the entry may be logged in the Hand-off Log. The type of hand-off may also be selected by the sending provider at the time of the hand-off, and this information may be added to the Hand-Off Log (such as “Overnight Hand-Off,” or “Weekend Hand-Off”) 154. The clinical content of the hand-off that was communicated electronically from one provider to the next may be viewed retrospectively 156.

Viewing the log 144 may allow for a clear, chronological display of what provider cared for a patient at any particular time. Coupling of real-time provider hand-offs to such a log results in more accurate information about the assigned provider for each patient. Existing EHRs are frequently incorrect regarding what provider is actually caring for a patient. Practice groups rely heavily on e-mails, spreadsheets, word documents, or other tracking documents to assign patients, and these are not a formal part of an organization's EHR. Correspondingly, EHRs may rely on a provider's entry of a separate order to change the caring provider, something which may or may not be done in a timely fashion, and which sometimes is not completed at all. Some EHRs depend on interfaces from a separate registration system; this setup inherently may result in inaccuracies in identifying who the caring provider is, due to delays, downtime, or by nature of the interface and asynchronous information. Other EHRs require manual verification of an order to change the provider, resulting in additional delays and, in some cases, no action upon the order at all. Further, if one provider enters an order to assume care as the attending provider (the “attending provider” is the provider who is actively caring for an inpatient), and the patient is shortly thereafter reassigned to another attending provider (within minutes or hours), if the subsequent change order is not entered or verified, then there may be a caring provider logged in the EHR that never actually cared for the patient at all. In embodiments, the Hand-Off Log may be coupled to the designation of a receiving provider as the “attending physician.” When one provider completes the template information in a hand-off form, the log 144 is the most accurate way that can be used prospectively to accurately identify the correct attending physician or responsible provider. Multiple hospital staff may use this information to most accurately identify the provider who is responsible for a particular patient (such as unit coordinators, nurses, and other providers). This proper role identification may avoid incorrect pages or calls to providers who are not-responsible for a patient's care, and may help to avoid delays in care when a patient requires an evaluation, diagnostic test, or therapy which needs to be ordered by the responsible provider. By tracking unique hand-offs which are not currently documented or auditable in a hospital EHR, such as hand-offs to a temporary covering provider during an overnight period or weekend period, the hand-off log becomes the most reliable way to reconstruct all transitions of care that occurred during any particular hospital stay. By designating the correct caring provider, the Hand-Off Log also helps hospital staff anticipate who will be caring for a patient. For example, a patient in the emergency department may be under the care of an emergency department provider; however, hospital staff may need to quickly identify the caring provider during or near the time of transfer to a hospital unit. If the patient's transfer from the emergency department is delayed, the patient may remain in the emergency department but may transfer to the care of the new caring provider. The Hand-Off Log allows for easy identification of who is the caring provider regardless of location. EHRs may have built-in rules that assign a caring provider based strictly on location. Hospital locations may operate using different EHRs; the Hand-Off Log, through interfaces with multiple software platforms, may harmonize the designated caring provider among distinct clinical systems, allowing for accurate identification of the caring provider regardless of patient location, electronic clinical system, or user.

In embodiments, the Hand-Off Log may be used to reconstruct what provider was caring for a patient at any particular time retrospectively. EHRs frequently have a single “attending physician” or other related provider data field, the contents of which is replaced with each update. This results in data loss and allows capture of only the last person to enter an order to change this data field. The above-described limitations regarding the typical internal processes which govern who cares for what patient, and the possibility that users never enter the corresponding order in an EHR, makes for another reason that reconstructing this information is not easy and sometimes not possible at all with existing EHRs. Use of the Hand-Off Log may augment an organizations patient safety and quality program by more accurately identifying the correct provider to which care at a particular time should be attributed. Further, since the hand-off log enables viewing of the clinical content from any particular hand-off, through the view function 156, communications that took place between providers can be reconstructed retrospectively. Such communications, which usually occur via e-mails or through word processor documents and excel spreadsheets, are converted into an immutable log of clinical transitions of care and their associated content, through the hand-off log.

The Hand-Off Log also provides an opportunity to track attribution of non-attending physicians, prospectively and retrospectively. Non-attending providers, such as consultants that are involved in the care of a patient in the hospital (such as a cardiologist or surgeon) become involved in a patient's care by request and then provide their impressions and recommendations in the medical record through documentation. EHRs do not provide a way to track which consultant is involved in the care of a patient; this occurs through verbal conversation and can be supported by hospital policies and procedures. It is not possible to prospectively or retrospectively identify, through the medical record, what consultant is responsible for the particular medical issue for which their specialty is involved, without looking at signature's/authorship on written or electronic notes or medical orders. It is further not possible to determine if a hand-off from one consultant to another occurred at all. This is only evident in an EHR when a new consultant documents in the medical record, at which point they associate their name with the patient and medical problem. The actual hand-off may have occurred hours or days earlier through a phone conversation or e-mail. The Hand-Off Log enables a prospective understanding, grounded in an auditable database, of what consultant is involved in the care of a patient at what time.

In embodiments, the Hand-Off Log may be used for service placement for a patient who has been admitted to the same provider group in the past. Service placement choices may be appropriately impacted by considering what provider has cared for the patient in the past; providers who “triage” admissions to a service can view the hand-off log in real-time during the service's triage process. The log may be viewed during the triage process, and may serve as a point-of-decision tool to understand the identity of the actual last provider who cared for a patient. For example, an inpatient provider may choose to direct the patient to the care of a specific inpatient provider who previously cared for the patient; this promotes continuity so that a patient may be cared for by the same provider during another admission.

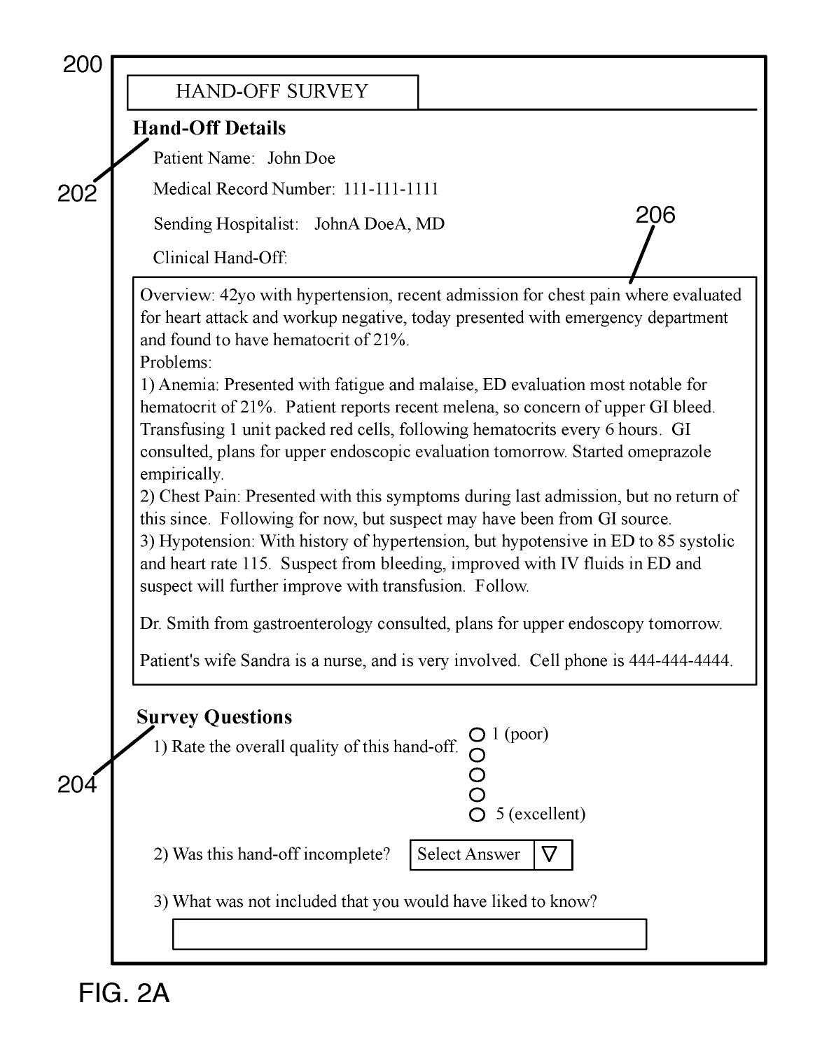

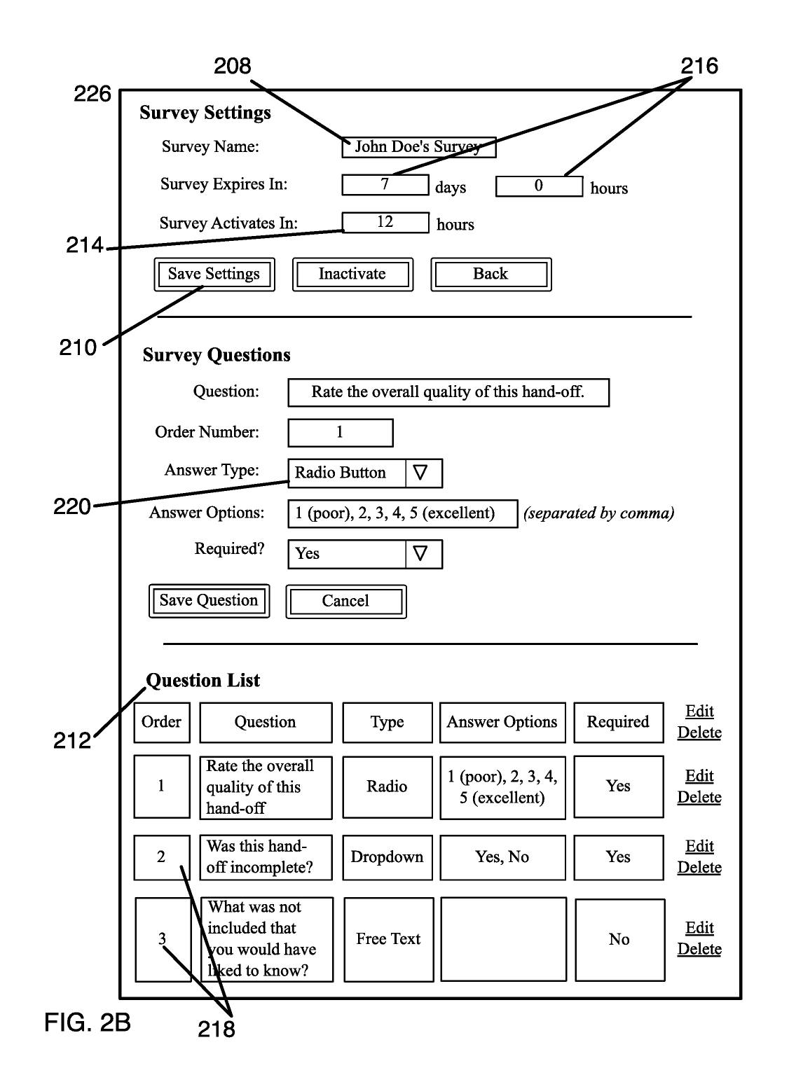

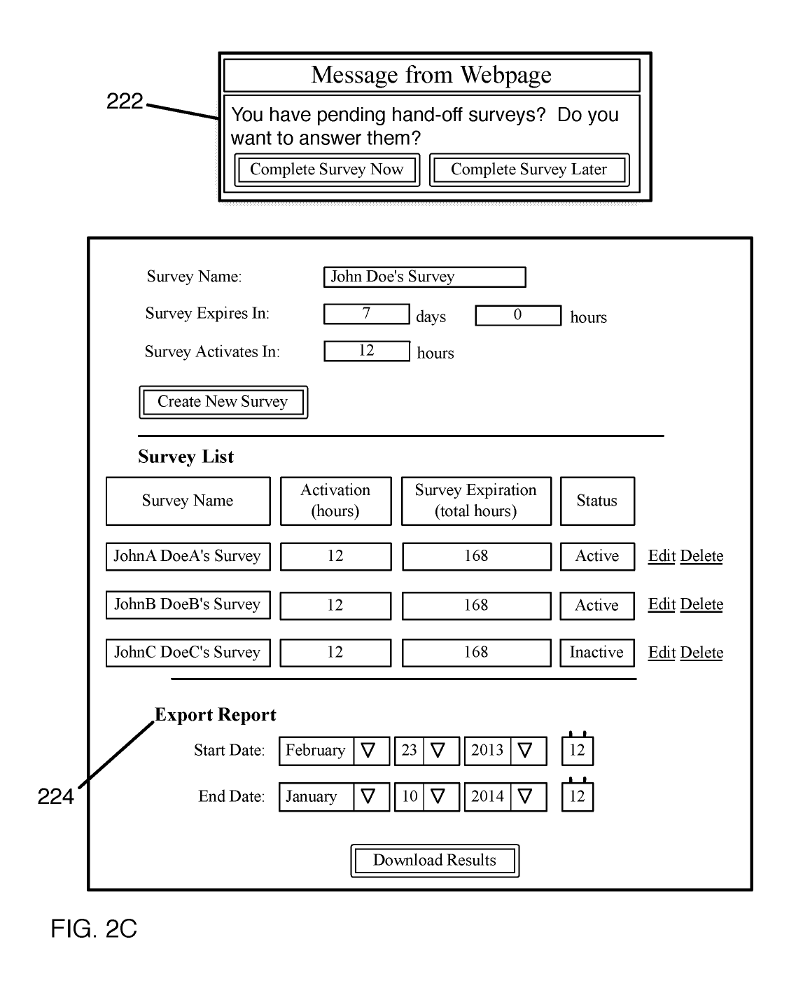

Embodiments of the systems and methods disclosed herein may comprise a hand-off survey module. In embodiments and referring to FIG. 2A, FIG. 2B, and FIG. 2C, the hand-off survey module may comprise functionality providing a front-end administrative interface 226, where hand-off surveys are created and where questions are formed. When a hand-off is entered utilizing the structured hand-off form provided by a healthcare workflow/EHR management platform, a hand-off log entry is created, as described above 202. When a hand-off log entry is created, this may result in the creation of a new survey question in a survey database, based on the survey specification created in the front-end survey administrative interface 226. The provider who receives the hand-off has the hand-off survey 200 displayed and may then complete the survey questions 204. The survey 204 may be displayed beginning at the specified time (as specified in the administrative interface). In embodiments, the survey 204 may stop being shown to the receiving user after the expiration time specified in the administrative interface 226. The survey that is displayed to the receiving provider may contain the clinical information that they received at the time of the hand-off 206, so they can most accurately rate and comment on the quality of the hand-off, among other reasons. In embodiments, survey results may be exported at any time by the administrative user. In embodiments, survey results may be exported in the form of a report 224.

In embodiments, the hand-off survey module may comprise a survey database. An administrative user may export the survey database. In embodiments, the survey database may comprise fields such as, but not limited to, patient name, patient medical record number, survey type ID, survey ID, when the survey was activated, date/time survey was first shown, date/time survey was completed, date/time survey expired, sending user, receiving user, status of the survey (completed or expired), question setting (optional or required), question content, answer content, and hand-off content/synopsis (the clinical information that was entered at the time of the hand-off), among others. In embodiments, administrative users may create one or more surveys. In embodiments, all surveys may be required to include options such as the name 208, whether the survey is active/inactive 210 (whether survey will trigger the creation of hand-off surveys), survey questions 212, when the survey is to be first displayed 214 (how long after a hand off the survey will be displayed to the receiving provider), and the date when the survey expires 216. In embodiments, more than one question may be added to a survey 218. In embodiments, questions may be generated in several available formats 220, such as, but not limited to, a radio button, checkbox, dropdown selection, or a free text response. In embodiments, the healthcare workflow/EHR management platform may comprise providing notifications to remind a user to complete a survey 222. A receiving provider may choose to ignore the reminder or accept the reminder. In embodiments, reminders may be displayed multiple times, if ignored.

In embodiments, the Hand-Off Survey Module draws from the above-described Hand-Off Log. By doing so, the survey is accurately attributed to both sending and receiving physicians. This is significantly superior to current EHR attribution capabilities (due to significant limitations reviewed above). In embodiments, the Hand-Off Survey Module links the sending and receiving provider information with a provider assessment of hand-off quality through automated logic, which works off a provider's hand-off workflow. This logic allows for assessment of hand-off quality in a way that is automated while avoiding negative impacts on provider workflow. Currently, publications on hand-off quality depend on sending a survey manually to a provider; information related to the hand-off is not presented in-line with the survey. Further, auditing hand-off content retrospectively is not possible, due to either mutable fields in an EHR, or, more commonly, due to the use of personal e-mails or word documents to capture hand-offs. The below-described immutable Hand-Off Threading captures hand-offs prospectively through integration into the survey tool at the time of the hand-off.

In embodiments, export of the Hand-Off Survey results may allow for data analysis of hand-off features and hand-off quality through review of real-time hand-off content or through analysis of survey question response data. Hand-off content or survey analytics may contribute to several areas that are receiving increasing interest by the medical community, including: (1) Individual physician hand-off quality performance, which is a Joint Commission area of focus and an increasing focus of physician groups and organization. Assessing a physician's quality of care can include process or outcome measures; this represents a new way to measure a process measure that is recognized as critically important. Automation of these surveys provides for accumulation of enough data to draw conclusions on a particular provider; (2) Measurement can be used for pay-for-performance goals (through transmission of audit results to payers), individual provider bonus (through integration in an organizations healthcare quality and safety database), for provider compliance (through integration or reported to an organization's credentialing database), or for research (by integration into tools that provide statistical analytic capabilities); and (3) The survey can be structured to include a specific question that targets any group or organization's performance goals. For example, if a hospital signs a quality-risk contract which includes reduction of urinary catheters as a target measure (linked to a financial incentive), then the hand-off survey can be modified to include a question regarding the appropriateness of urinary catheters. This would be integrated into every hand-off in the organization, and would allow the hospital to measure baseline data, track performance over time, and provide feedback to offending providers.

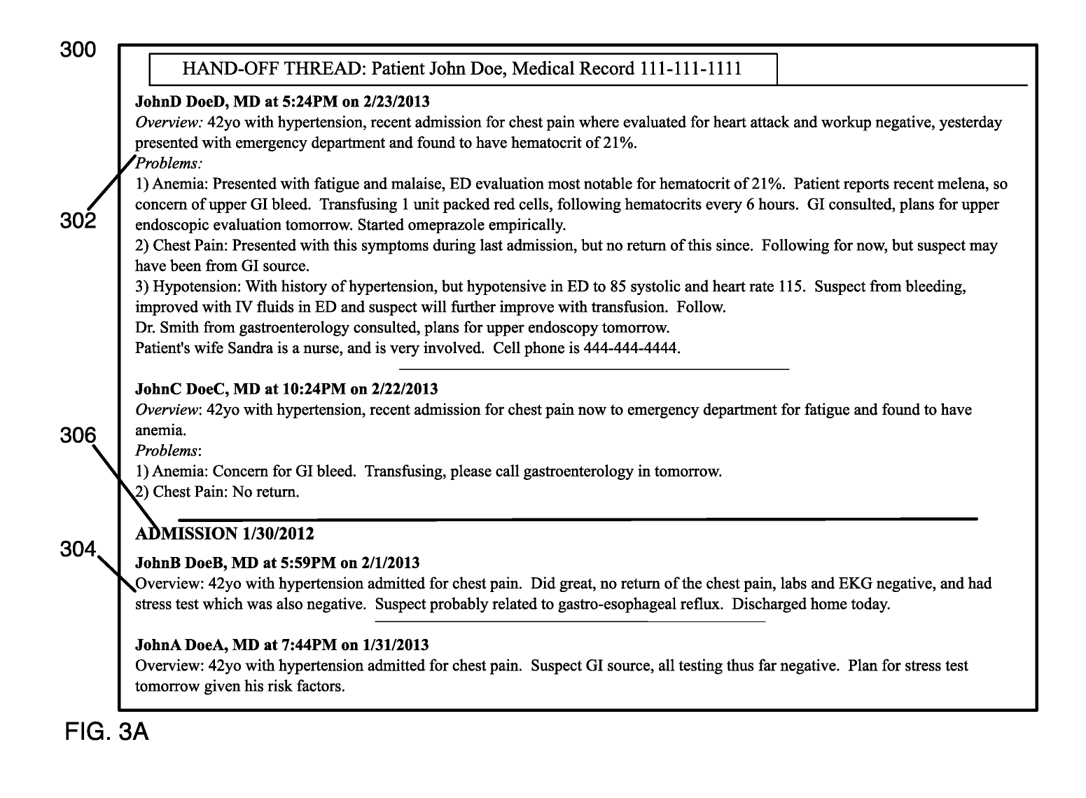

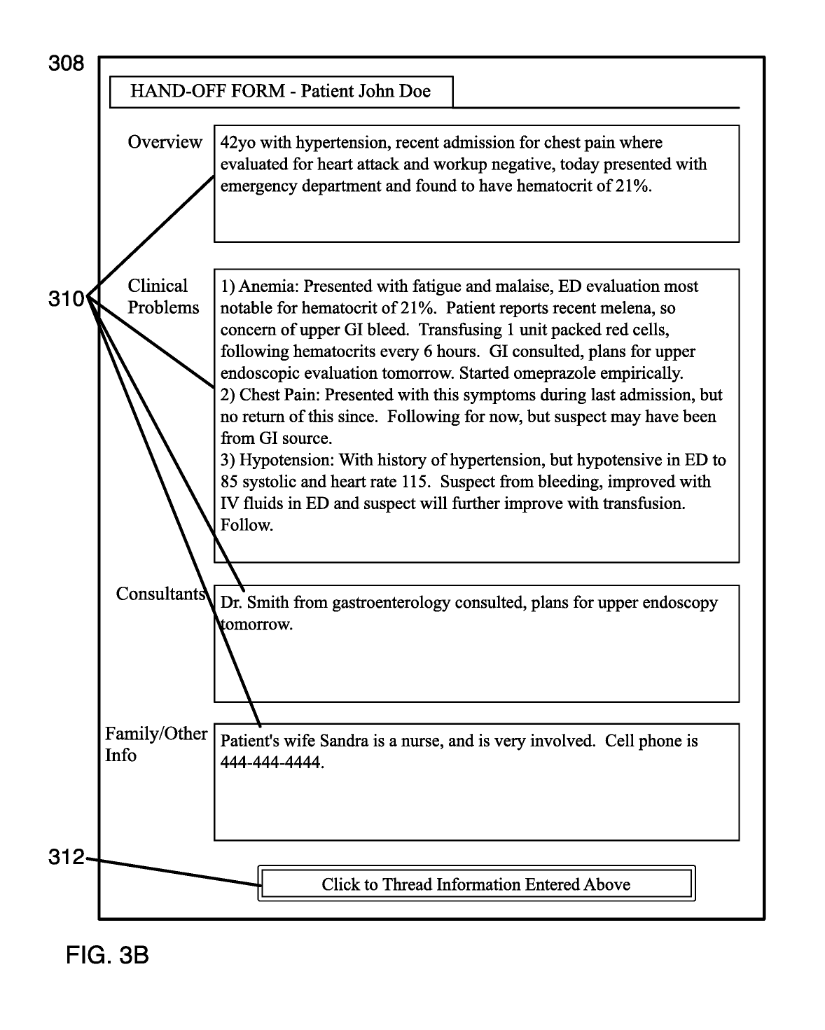

Embodiments of the systems and methods disclosed herein may comprise a hand-off threading module. In embodiments and referring to FIG. 3A and FIG. 3B, the hand-off threading module may comprise functionality allowing for the creation of an information thread of hand-off content 300. In embodiments, a user may enter a hand-off using the structured hand-off form 308. The information is entered into the structured hand-off form clinical synopsis fields 310. These clinical synopsis fields 310 may comprise information such as, but not limited to, the clinical issues a patient is experiencing and the plan to treat the patient, among others. A user may then activate a function 312 within the structured hand-off form via the user interface to add the entered clinical information 310 into a thread 300. This may result in date/time stamping and user-stamping of the clinical information 302, along with other benefits to memorializing the synopsis 302. In embodiments, a final “Discharge Synopsis” 304 may be further added to the thread (the synopsis that is entered at the time of discharge) in addition to the hand-off synopses 302. Users may then be able to view the full thread of hand-off information 300 by selecting a different function within the healthcare workflow/EHR management platform via the user interface. In embodiments, information from a prior inpatient encounter 306 may be displayed in conjunction with information from a current inpatient stay, so that the thread provides a unified location for all hand-off information. The thread allows for viewing information from the time of triage and admission (for when a patient is triaged for admission and then admitted), off-service (when a patient is handed-off from one provider to another), overnight (for temporary coverage), overnight events (when a temporary provider transitions care back to a daytime provider and enters overnight clinical issues that arose), and the time of discharge (when a patient is discharged), providing information clearly in a reverse chronological order in a concise format.

There exist several benefits to implementations of the above-mentioned embodiments. Standard clinical documentation (such as documented complete History and Physicals completed at the time of admission or Discharge Summaries completed at discharge) is intended to be highly comprehensive. A significant implication, however, is that clinical documentation is very lengthy and does not distill information to key points. Rapid retrieval, therefore, of key information is not the goal of standard clinical documentation. Threading key summary information using the Hand-Off Threading Module allows for rapid understanding of the central issues at key times. By viewing a thread of prior hand-offs, a user may be able to rapidly identify the main clinical issues and further understand how clinical issues for a particular patient evolve over time. Further, since hand-offs may be saved indefinitely, patients who are readmitted to the hospital may have hand-offs viewed from prior admissions as well. This may allow a user to quickly understand the medical problems that were present during prior inpatient encounters. Further, by viewing prior admissions' final/discharge synopses, users may quickly identify what the plan of care was at the time of discharge. This may be very helpful as patients frequently present with similar clinical problems; understanding plan of care during a recent admission may help direct the plan of care for the current encounter.

In embodiments, the Hand-Off Threading Module's date, time, and user-stamping of the clinical information is valuable as well since this feature allows for easy identification of a previous provider who added a thread. This may enhance communication with key individuals when questions arise. For example, a caring inpatient provider may easily identify a selected provider's synopsis from a prior admission and this allows the caring provider to ask the selected provider specific questions about the patient's medical conditions without having to sift through volumes of EHR medical records from a prior encounter. Similarly, if a caring provider has a question for an overnight, temporary covering provider who threaded clinical information, the caring provider can easily identify the correct provider with whom they should communicate to get further information.

In embodiments, the Hand-Off Threading Module includes user-stamp and time/date stamp information. This positions a provider to easily identify who cared for a patient during a prior admission. This may help providers understand who may be best to direct questions to, if issues arise or clarification needed.

In embodiments, the Hand-Off Threading Module enables capture of information at several key moments in time which, while very important for a patient's hospitalization, are not able to be recreated using information from the EHR, or can only be recreated with great effort after reviewing multiple documents from a hospital EHR. For example, when a provider in the emergency department determines that a patient requires admission to the hospital, they may contact an admitting inpatient provider. The emergency department clinician may document a comprehensive emergency department visit note, and the admitting inpatient provider may document a separate, comprehensive history and physical admission note. Neither the emergency department note nor the history and physical may contain the information communicated from the emergency provider to the admitting provider. Each provider may document their own impression and plan, however critical-decision making that occurs in the interface of that dialogue, the triage clinical synopsis, would not be captured anywhere in these separate clinical documents. Landmark moments in the care of a patient are, therefore, not captured in traditional clinical documentation but are important in order to most fully comprehend and reconstruct a patient's clinical course. These key times, which are not captured due to the nature of existing standard clinical documentation, are distinctly organized and logged by the Hand-Off Threading Module. The Hand-Off Threading Module is setup to log clinical information at defined times, which constitute (1) a triage synopsis, (2) a synopsis at the end of each day, whereby a caring provider hands-off to a temporary covering provider, (3) other moments in time when one a covering provider is temporarily covering for the caring provider such as prior to weekends or holidays, (4) after the temporary covering physician completes their coverage duties, whereby they use the threading to briefly document and then communicate changes in the patient's condition back to the caring provider, (5) a discharge synopsis, (6) a synopsis at any other critical points during a patient's hospital course, such as when there is a major change in medical condition.

In embodiments, the Hand-Off Threading Module is easily accessible from any device, such as desktop computer, handheld device, or tablet; information can be viewed and hand-off threads can be added from any of these devices. In embodiments, the threading of the clinical information enables the system to pre-populate fields for the next update of these respective fields which allows for provider efficiency gains.

In embodiments, the Hand-Off Threading Module generates threads that are automatically communicated to external medical providers in independent offices; this therefore keeps providers in independent offices and different medical information systems up to date on the progress of a patient who is in the hospital. Providers may include the Primary Care Provider (or delegate) and/or Specialists (such as a surgeon or cardiologist, or their delegate). Each of the offices/organizations of these providers may be independent, and communication of the threaded clinical information can occur through encrypted e-mail or via interfaces with other EHRs and associated clinical messages. Threads can also be integrated with a provider or healthcare organization's EHR to allow for retrieval as a clinical note or document.

In embodiments, the Hand-Off Threading Module may enrich a hospital or a practice's quality improvement efforts, at the individual or organizational level. This benefit emerges due to the ability to recreate a provider's understanding of the clinical facts at one of the several above-mentioned key times. Threading, therefore, enables retrospective construction of events that is not possible with current EHRs. In addition, the above-noted key times, which are landmark points in a patient's inpatient course, are important to retrace in order to contribute to an understanding of what may have gone wrong and how care could have been provided in a more optimal fashion.

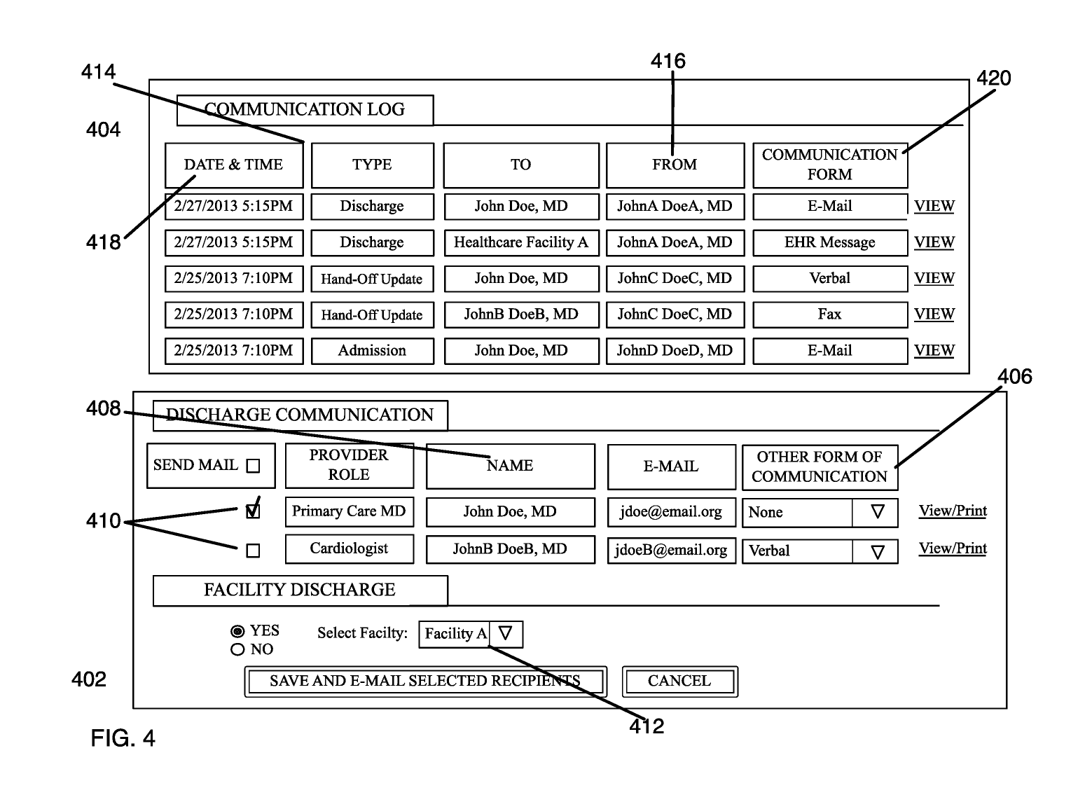

Embodiments of the systems and methods disclosed herein may comprise a communication and logging module. In embodiments and referring to FIG. 4, the communication and logging module 402 may comprise functionality allowing the entering of an admitting clinical synopsis for a patient when that patient is admitted to the healthcare providing facility. The synopsis may be entered into the healthcare workflow/EHR management platform. In embodiments, the admitting clinical synopsis may be sent to one or more outpatient providers, so that outpatient providers may be informed that the patient is admitted, and receive a brief overview of the reason for admission. In embodiments, when a provider-to-provider hand-off occurs, the clinical synopsis at the time of the hand-off can again be sent to selected outpatient providers, so that they can be informed with an update. In embodiments at the time of discharge, selected outpatient providers may receive a final update, where the clinical synopsis detailing the plan of care at discharge is communicated. In embodiments, communications from the communication and logging module 402 may occur electronically by means such as, but not limited to, email, and may be included in the Communication Log 404. The Communication Log 404 may provide a list of what communications are sent. In embodiments, when an outpatient provider's contact information is not available, the communication and logging module 402 may allow users the option to select an alternative form of communication 406, so that they can log that communication with selected outpatient providers has occurred in alternative ways, such as, but not limited to fax or telephone, among others. In embodiments, users may select the individual to send communications to 408. Users may additionally add an unlimited number of ambulatory or inpatient providers with whom to communicate 410. In embodiments, users may be able to select the method in which they wish to send the communication 406. In embodiments, users may have the option to communicate with providers at other specified non-ambulatory facilities 412. In embodiments, the Communication Log 404 may contain a plurality of data elements, such as, but not limited to: (a) the type of communication (admission, hand-off, or discharge) 414, (b) which user sent the communication 416, (c) date/time of communication 418, and (d) the form of communication 420, among others (collectively, as used herein “metadata”). For communications that occur electronically (such as e-mail), a user may view the content of the electronic communication by selecting on the specific entry in the communication log.

In embodiments, the communication and logging module may integrate communication with ambulatory providers in the context of the admitting and triage process, hand-off process, and discharge process. By integrating communication into the inpatient caring provider's workflow, a caring provider is not required to separately communicate with ambulatory providers. Updates to the synopsis at the time of admission, hand-off, and discharge are instead simply sent to the ambulatory care team, thereby reducing workflow redundancies and making the frequency and regularity of communication more robust. Updates may be entered, communicated, and logged from any device, such as desktop computer, handheld device, or tablet. This intuitive, accessible, and unified communication log is superior to existing technology. EHRs are non-uniform and highly inconsistent with respect to communication tracking. Examples of non-uniform methods that EHRs may use to track communication may include free text in a discharge summary, written text in a daily progress note, or an order in a computerized order entry system. EHRs may include integrated clinical messaging, however that only accounts for communication performed using the EHR-integrated clinical messaging tool.

The Communication Log 404 may allow users to keep track of all of their communications, through immutable recording of critical information, allowing them to ensure that they have communicated to all necessary providers. The Communication Log 404 may also allow users to audit communications, to ensure that communication is occurring consistently. Administrative auditing of a multitude of patients' communication logs can be used to track as a quality performance indicator per provider or group; the ability to track such granular and detailing communication information is a novel quality measure for physician and non-physician providers. Further, auditing can be used to contribute information for the peer review process when a question arises regarding communication. Failure in communication is consistently sited as the most common cause of medical errors and medical malpractice lawsuits. Conversely, EHRs are auditable only through manual reconstruction from multiple sources. Specifically, one would need to reconstruct communication activities from an EHR by sorting through computerized order audit trails, free text in paper or electronic notes, and possibly by reviewing personal e-mail account items or clinical messaging audit trails.

In embodiments, the systems and methods disclosed herein may comprise a readmission notification and survey module. In embodiments, the readmission notification and survey module may work in conjunction with the hand-off log module to clearly identify what hospitalists were involved in a patient's care for a prior hospital stay. The uniquely accurate Hand-Off Log accurately identifies the discharging physician and readmitting provider. Then, when a patient is readmitted during a specified time frame, the hospitalist(s) involved in the patient's care during the last hospital stay may be presented with a notification via the readmission notification and survey module, making them aware that the patient was readmitted. The users may then have the opportunity to comment on the contributing factors for the admission in a readmission survey. When a readmission occurs, discharging and readmitting providers may receive a notification alert through an automated message that a readmission occurred and that they have a survey to complete. These messages can be integrated with EHR clinical messaging, an organization's e-mail server, an organization's paging/texting technology, or with an automated alert system.

In embodiments, readmission notifications and readmission surveys may include the final, discharge clinical synopsis from the last hospital stay (from admission #1) and readmission synopsis (from admission #2), allowing for a concise reminder to the discharging physician of the medical issues, treatments and plans when they discharged the patient, and showing them a concise summary of why the patient was readmitted. Displaying highly focused and key information optimally positions both discharging and readmitting providers to complete a survey related to the preventability of the readmission. Surveys also may display communication information (from the communication log described above), enriching the survey by adding another dimension to the assessment of the readmission. In embodiments, the hospitalist who performs the readmission may also receive a notification that the patient was readmitted, and also can comment on the reason, from their perspective, for the readmission. This additional feedback may enrich readmission data and may ultimately allow for a better understanding of what factors may have contributed to a patient's readmission. By querying all involved providers about a readmission, providers are forced to always carefully consider factors contributing to admissions, and may help to increase attention to certain factors that may be associated with a high risk for readmission. Traditional EHRs are only capable of presenting traditional, comprehensive clinical documentation. Discharge summaries and history and physical documents are lengthy and contain unrelated, boilerplate language to fulfill compliance and regulatory requirements; traditional EHR documentation cannot realistically be read in depth at time of survey completion.

In embodiments, accessibility of surveys, as well as the most relevant, in-line clinical information, is critical to ensure that the surveys are completed, and that completion occurs very close to the moment of readmission. This innovation allows for web-based access of the surveys, from any browser and from several devices (such as desktop computer, handheld device, tablet). This technology can be easily coupled with a group or organizational expectation to check for any new readmission surveys on the software while a provider is off service, so surveys can be completed within a few hours or days. Accessing the full medical record to complete a readmission survey is not necessary with this innovation. Further, providers may hand-off frequently, and often may not work for several days in a row after a period of time on service. They further have no reason to access any medical records when home or away from work in another location; waiting to complete a paper survey inserted into their mailbox or e-mailed to them until they return to the hospital to view the medical record results in delays in survey completion where events of the hospital stay are no longer fresh in their mind.

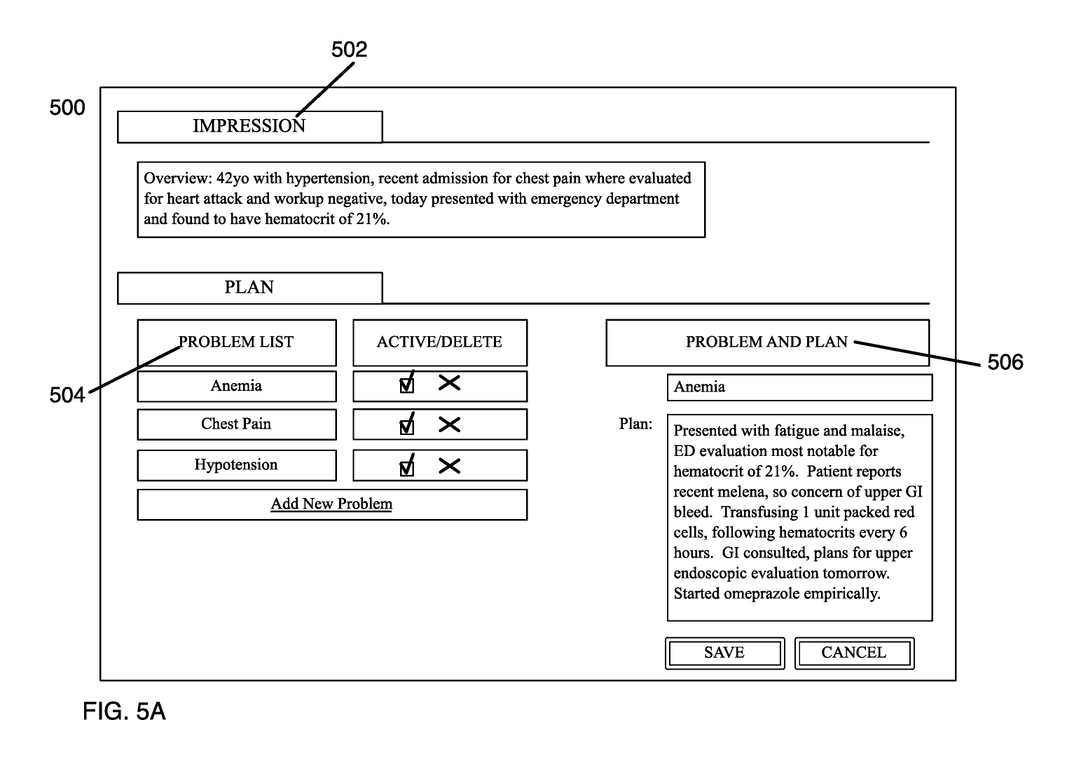

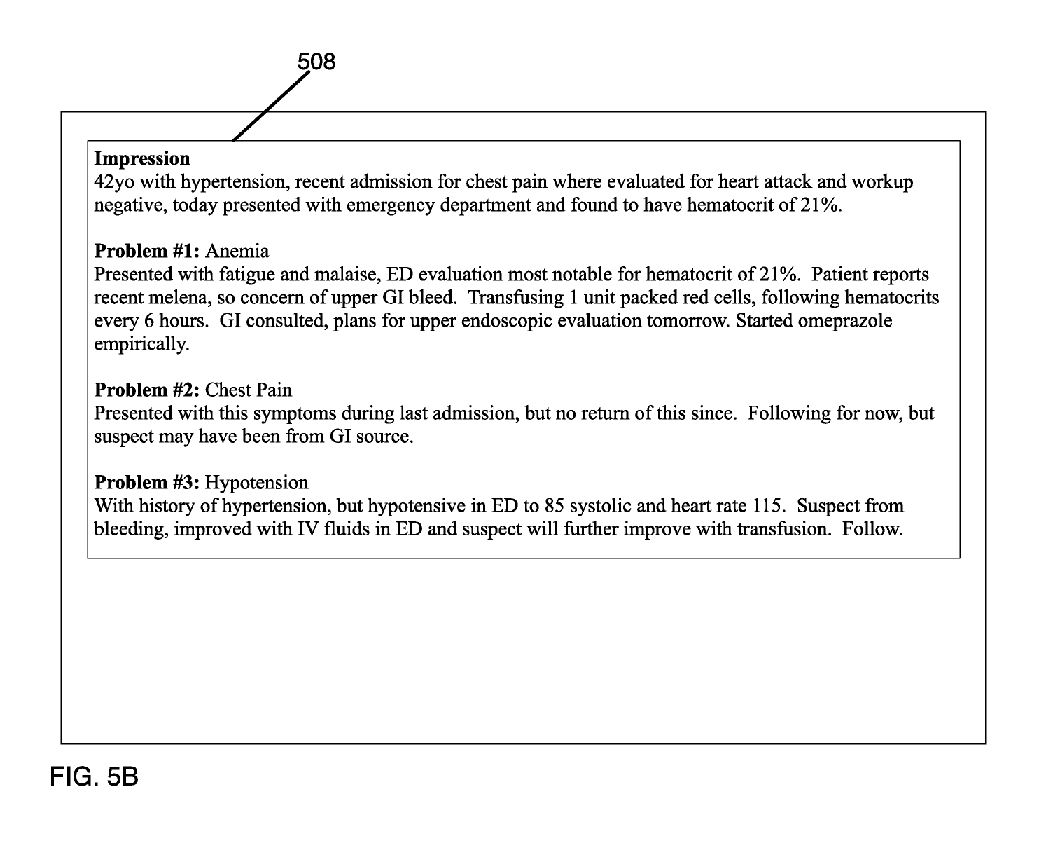

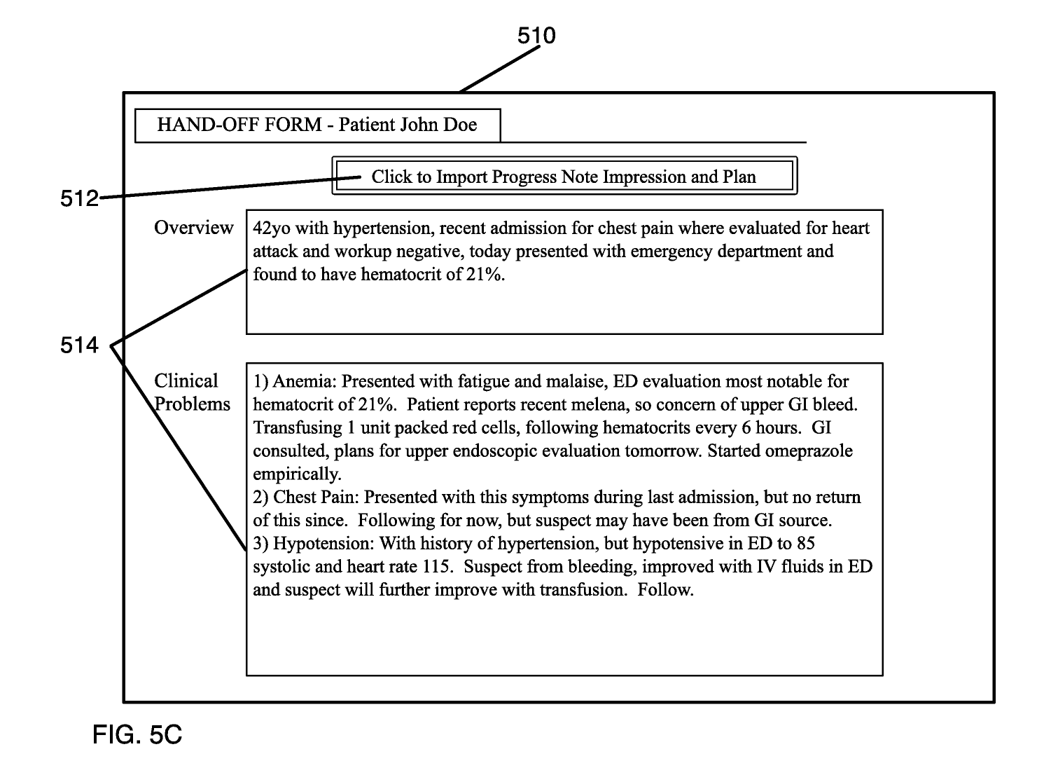

In embodiments and referring to FIG. 5A, FIG. 5B, and FIG. 5C, the systems and methods disclosed herein may comprise a progress note creation tool 500, where a clinical impression 502, problems 504, and plans 506, among other data, for each respective problem is logged by the user. The progress note creation tool 500 may generate a provider's documentation/progress note 508, which may be generated by completing fields within the progress note creation tool 500. The systems and methods disclosed herein may comprise a hand-off auto-import module 510. The hand-off auto-import module 510 may comprise functionality that allows a provider, when the provider goes off service, to automatically import the “Impression” 502 (impressions of a patient's clinical status and progress) and “Plan” fields 504 506 (reviews of each medical problem and corresponding plans to address the problem) from a provider's progress notes 508 into the hand-off form. In embodiments, the hand-off auto-import module may be accessed in conjunction with the hand-off form. A user may access a function activating the hand-off auto-import module 512 in the healthcare workflow/EHR management platform via the user interface. The function 512 may activate the hand-off auto-import module, thus automatically completing the hand-off clinical content fields 514 with data from impression 502 and plan fields 504 506 from that day's progress note 508.

In embodiments, the hand-off auto-import module sets hand-offs up to be highly focused summaries that allow a provider who is assuming care of the patient to rapidly understand key issues. By importing the clinical impression, list of problems, and plans, the receiving or covering provider can begin to develop their own impression and make important clinical decisions by reading this summary of information. Quick review of key clinical issues is very important, as clinicians frequently have multiple competing demands; requiring a busy clinician to sift through a patient's medical record to distill key issues is a suboptimal use of time and may delay care to patients. Progress notes, however, are comprehensive documents that review the patient's symptoms, clinical progress, physical examination, laboratory and radiologic data, and the provider's impression and plan. All of this information is not required for the hand-off. Instead, an effective hand-off should contain a summary of a patient's medical problems and planned diagnostics and therapies. Existing EHRs risk cluttering hand-offs with so much information that it may take a long time for a provider to understand the key clinical issues, while not assisting providers with the aspects of a hand-off that require critical-thinking, such as the clinical rationale pertaining to a particular therapy. The hand-off created by the hand-off auto-import module assists the caring provider by importing their high-level clinical impressions, and accompanying critical thinking, in the hand-off form. When a provider needs to know more than is provided on the hand-off, such as an inpatient medication list or allergies, they can easily navigate to targeted locations in the medical record.

In embodiments, the hand-off auto-import module enables providers to import information from an already documented progress note. The progress note is already a very complete document, detailing the patient's clinical status and plan for diagnostic evaluations and therapies. Providers are required to safely transition the care of their patients to nighttime coverage, weekend coverage, and/or coverage when they go off service. The hand-off auto-import module streamlines workflow by avoiding the redundancy of typing a progress note and also typing a summary of the patient information to hand-off to another provider. Provider groups or healthcare organizations may draft their progress note in an existing organization's EHR. Use of the Hand-Off Auto Import Tool can extend to this situation, where selected hand-off information would be populated from the organization's EHR-based progress note to the OnServiceMD Hand-Off Form and relevant fields. Provider groups or healthcare organization may setup their hand-offs in such a way that they use an organization's existing EHR. Use of the Hand-Off Auto Import Tool can also extend to this situation, where progress notes documented in OnServiceMD can have data from selected fields flow to the EHR hand-off fields.

In embodiments, the above streamlined process made possible through the auto-import tool relates not only to hand-offs from one provider to another while a patient is in a non-ambulatory setting, but also pertains to workflow when a patient is transitioned to another facility. Auto-Import of the impression and plan for key clinical issues allows for streamlining of workflow when a patient is, for example, discharged, and also helps to ensure completeness of the transition of care (that all issues are included and the key aspects of each issue are mentioned). A “final” hand-off, which is shared with an accepting outside provider (such as accepting provider at a nursing home, or a primary care doctor when a patient is discharged home), may be sent electronically to the next provider through encrypted e-mail, an EHR integrated clinical message, or through fax or other means of communication. Therefore, use of the hand-off auto-import module with transitions of care streamlines inpatient provider workflow, optimizes transitions of care, and, when linked with the communication log, is tracked and auditable.

In embodiments, the Hand-Off Auto Import Tool may enhance the safety and quality of the hand-off. Parallel processes where a provider documents a progress note and then separately drafts a hand-off is not only inefficient, but also may result in incomplete information. A busy clinician, after writing a comprehensive progress note, may not remember to include the multitude of issues in the hand-off, which is usually completed at a later time of the day using a separate process. Even when all medical problems are included in the hand-off, a provider may fail to mention an important feature of one of the problems. Therefore, linkage of the progress note's impression and plan to a hand-off ensures that important problems are not excluded and helps to ensure that a caring provider includes all pertinent information in their hand-offs.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

The methods and systems described herein may be deployed in part or in whole through a machine that executes computer software, program codes, and/or instructions on a processor. The present invention may be implemented as a method on the machine, as a system or apparatus as part of or in relation to the machine, or as a computer program product embodied in a computer readable medium executing on one or more of the machines. In embodiments, the processor may be part of a server, cloud server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor, or any machine utilizing one, may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, and so forth, or any combination of these, and all such implementations may be within the scope of the present disclosure. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps associated therewith, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine-readable medium.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

The use of the terms “a” and “an” and “the” and similar referents in the context of describing the disclosure (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure. All documents mentioned herein are hereby incorporated in their entirety by reference.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

What is claimed is:

1. An electronic health records (EHR) management platform running on a server, the platform comprising:
a communication module that records and timestamps communications occurring during an admittance of, a stay of, and a discharge of a patient at an inpatient healthcare facility, automatically generates a communication if a predetermined event occurs, and generates a communication log of the communications;
a hand-off logging module that records hand-off information relating to a hand-off of the patient from receiving health services in the inpatient healthcare facility from a first healthcare provider to receiving health services from a second healthcare provider, wherein the hand-off logging module further records, during the stay of the patient at the inpatient healthcare facility, hand-off information relating to any subsequent hand-offs of the patient to other healthcare providers, wherein the recorded hand-off information includes corresponding names of the healthcare providers providing health services to the patient and a time stamp associated with each hand-off, wherein the hand-off logging module further generates a hand-off log including the recorded hand-off information and the patient's clinical information,
wherein a communication to a corresponding discharging healthcare provider is automatically generated by the communication module if a readmission of the patient to the inpatient healthcare facility occurs;
a hand-off threading module that receives a respective clinical synopsis relating to the patient from each named healthcare provider associated with a recorded hand-off at the time of hand-off, including a clinical synopsis by a discharging healthcare provider, wherein each clinical synopsis comprises a status of the patient, a progress of the patient, and a proposed care plan, wherein the hand-off threading module generates a hand-off thread that includes each clinical synopsis, including the clinical synopsis by the discharging healthcare provider and a clinical synopsis relating to any prior admission of the patient to the inpatient healthcare facility; and
a user interface that enables a user to view each of the patient's hand-off log, the patient's communication log, and the patient's hand-off thread including information regarding any prior admission.

2. The EHR management platform of claim 1, further wherein the communication module is enabled to send communications regarding at least one of the patient's hand-off log and the patient's hand-off thread to at least one of an external healthcare provider, an external electronic health records system, and a quality monitoring module.

3. The EHR management platform of claim 1, wherein the patient's hand-off log also includes patient discharge information.

4. The EHR management platform of claim 1, further comprising a progress note creation tool for recording a progress note of the patient by a named healthcare provider.

5. The EHR management platform of claim 4, further comprising a hand-off thread export tool for exporting the recorded progress note of the patient to be incorporated in the hand-off thread.

6. The EHR management platform of claim 1, further comprising a survey module that generates surveys, wherein each survey relates to a specific hand-off of the patient in the healthcare facility and a respective quality of the specific hand-off, and each survey includes corresponding specific hand-off information actually received by an intended survey recipient.

7. The EHR management platform of claim 6, wherein each survey is communicated to a corresponding healthcare provider who is on a receiving end of the specific hand-off.

8. The EHR management platform of claim 7, wherein the survey module records and analyzes survey results.

9. The EHR management platform of claim 1, further comprising a survey module that generates a survey regarding readmission of a patient.

10. The EHR management platform of claim 9, wherein the readmission survey is directed to a discharging healthcare provider.

11. The EHR management platform of claim 1, wherein the healthcare providers are medical doctors.

12. The EHR management platform of claim 1, wherein the healthcare facility is one of a hospital, a skilled nursing facility, a rehabilitation facility, a long-term acute care facility, and a post-acute care facility.

13. A method comprising:
recording patient clinical information associated with a patient's admission to and discharge from an inpatient healthcare facility, wherein the patient clinical information includes a respective time stamp of the patient's admission and discharge;
recording information relating to each hand-off of care of the patient in the inpatient healthcare facility from one healthcare provider to another healthcare provider, at the time of each hand-off, wherein the recorded hand-off information includes corresponding names of the healthcare providers providing health services to the patient, a time stamp associated with each hand-off, and the patient clinical information;
generating a hand-off log including the recorded hand-off information relating to each hand-off, admission, discharge, and the patient clinical information;
generating a hand-off thread that includes a respective clinical synopsis relating to the patient by each named healthcare provider associated with each recorded hand-off, including the clinical synopsis by the discharging healthcare provider and any clinical synopsis relating to a prior admission of the patient to the inpatient healthcare facility, wherein each clinical synopsis comprises a status of the patient, a progress of the patient, and a proposed care plan;
and
communicating the patient clinical information to a user, wherein each transmitted communication is entered in a communication log which includes the names of an associated sending healthcare provider and a recipient healthcare provider, a time stamp associated with the communication, and a type of the communication, and wherein a communication to a corresponding discharging healthcare provider is automatically generated if a readmission of the patient to the inpatient healthcare facility occurs.

14. The method of claim 13, wherein the user includes at least one of an external healthcare provider, an external electronic health records system, and a quality-monitoring module.

15. The method of claim 13, further comprising generating a survey regarding a quality of a specific hand-off and communicating the survey to the healthcare provider on a receiving end of the specific hand-off.

16. The method of claim 15, further comprising recording and analyzing survey results.

17. The method of claim 13, further comprising generating a survey regarding readmission of the patient when the patient is readmitted to the inpatient healthcare facility after being discharged by a discharging healthcare provider and automatically communicating the survey to the discharging healthcare provider.
